(19) United States
(12) Patent Application Publication
(10) Pub. No.: US 2023/0135108 A1
(43) Pub. Date: May 4, 2023

(54) CANNABINOID SALT COMPOUNDS AND METHODS OF MAKING SAME

(71) Applicant: Canopy Growth Corporation, Smiths Falls (CA)

(72) Inventors: Mahmood Azizpour FARD, Smiths Falls (CA); Ben GEILING, Smiths Falls (CA); Mohammadmehdi HAGHDOOST MANJILI, Smiths Falls (CA)

(21) Appl. No.: 17/642,658

(22) PCT Filed: Jul. 23, 2021
(86) PCT No.: PCT/CA2021/051027

§ 371 Date: Mar. 11, 2022

Related U.S. Application Data
(60) Provisional application No. 63/056,332, filed on Jul. 24, 2020.
(60) Provisional application No. 63/182,105, filed on Apr. 30, 2021.

(51) Int. Cl. C07D 311/80; C07C 211/27; C07C 63/64

(52) U.S. Cl. CPC C07D 311/80; C07C 211/27; C07C 63/64

(57) ABSTRACT

Disclosed herein are cannabinoid salt compounds formed by mixing an acidic cannabinoid compound with a base. According to embodiments of the present disclosure, the base is selected at least for having a low toxicity. Also disclosed are methods of preparing cannabinoid salt compounds. Further, the present disclosure provides use of the cannabinoid salt compounds as an intermediate for isolating an ultra-high purity cannabinoid from a cannabis plant material or extract thereof.

### CROSS-REFERENCE TO RELATED APPLICATIONS

This application claims priority to and benefit of U.S. Patent Application Ser. No. 63/056,332 filed on Jul. 24, 2020 and U.S. Patent Application Ser. No. 63/182,105 filed on Apr. 30, 2021, each of which is hereby incorporated by reference in its entirety.

### TECHNICAL FIELD

The present disclosure relates to cannabinoid salt compounds, methods of making same, and use of these compounds as an intermediate for isolating ultra-high purity cannabinoids.

### BACKGROUND

The cannabinoids contained in many medicinal and recreational cannabis products are often derived from cannabis plant materials or extracts thereof. Conventional methods for isolating cannabinoids from cannabis plant materials include at least: (i) an extraction step where the cannabis plant materials are submersed in a suitable solvent, and (ii) an evaporation step where the solvent is removed by volatilization leaving behind a crude extract containing the cannabinoids.

Supercritical carbon dioxide (scCO2) extraction techniques exemplify this approach. scCO₂ is a non-flammable solvent that readily extracts cannabinoids, and it is readily volatilized. Unfortunately, inducing CO₂ into the supercritical state requires high-pressure and/or large-volume apparatus that introduce considerable process complexities, costs, and safety concerns. Moreover, scCO₂ is not a particularly selective extraction solvent—it readily dissolves a number of non-cannabinoid components from cannabis plant materials (e.g. waxes, pigments, and cell-wall fragments). As such, many scCO₂-based processes for cannabinoid extraction require further downstream purification steps.

Another conventional approach isolate cannabinoids from cannabis plant materials uses hydrocarbon- and/or alcohol-based solvents. A number of hydrocarbon- and/or alcohol-based solvents readily dissolve cannabinoids without taking up significant quantities of waxes, pigments, cell-wall fragments, and the like. Unfortunately, retrieving cannabinoids from such solutions is often problematic. Solvent evaporation requires significant amounts of time, energy, or both, and many processes suffer from subpar solvent-recovery processes. Further, many cannabinoids form entrained mixtures with the solvent when they reduced to small volume/high concentration units. Accordingly, cannabinoid isolation by solvent evaporation introduces the potential for a number of undesirable outcomes—solvent incorporation into consumable products, difficult to handle materials (e.g. viscous oils instead of free-flowing solids), and/or the need for additional purification are all problematic.

For at least to the foregoing reasons, improved approaches for isolating high purity cannabinoids are desirable, including compounds and intermediates that may be used in such processes. In particular, methods that provide cannabinoids in sufficient purity to enable solid-form handling are desirable.

### SUMMARY

The present disclosure relates to cannabinoid salt compounds, methods of making same, and uses thereof.

In an aspect, the present disclosure relates to a compound of Formula 1:

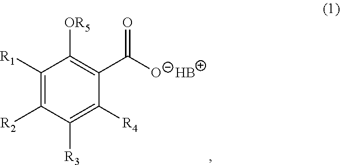

wherein R₁, R₂, R₃, R₄ and R₅ are, independently or in combination with one another, chosen so as to provide a cannabinoid, and B is a base that is of low toxicity.

In particular, the cannabinoid salt compounds disclosed herein comprise a base that is suitable for consumption by a subject, whether in a recreational or pharmaceutical preparation. In an embodiment, the base is benzathine. In an embodiment, the base is an amino acid, such as for example arginine, histidine, or lysine.

In an embodiment, the present disclosure provides a compound of Formula 2:

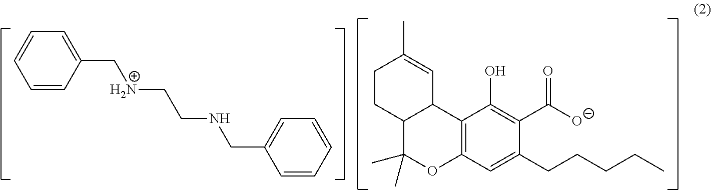

In an embodiment, the present disclosure provides a compound of Formula 3:

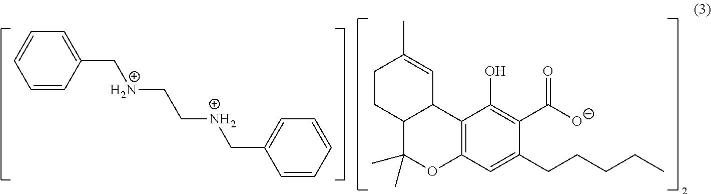

In an embodiment, the present disclosure provides a compound of

Formula 4:

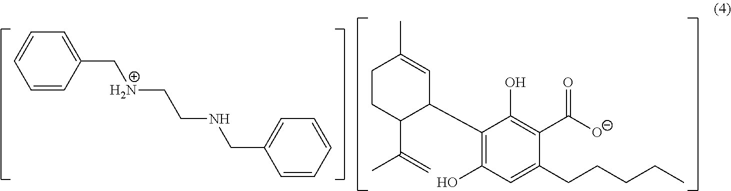

In an embodiment, the present disclosure provides a compound of Formula 5:

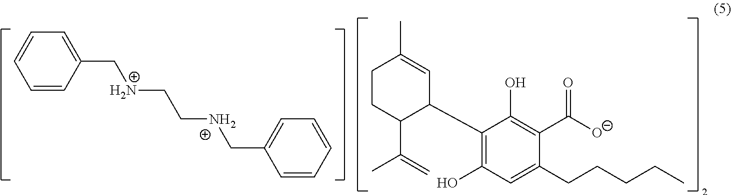

In another aspect, the present disclosure provides compositions comprising the compounds disclosed herein.

In another aspect, the present disclosure provides mixtures of different cannabinoid salt compounds.

In an embodiment, the present disclosure provides a mixture comprising a combination of any of the compound of Formula 2, the compound of Formula 3, the compound of Formula 4 and the compound of Formula 5.

In an embodiment, the present disclosure provides a mixture comprising the compound of Formula 2 and the compound of Formula 3.

In an embodiment, the present disclosure provides a mixture comprising the compound of Formula 4 and the compound of Formula 5.

In an embodiment, the present disclosure provides a mixture comprising the compound of Formula 2 and the compound of Formula 4.

In another aspect, the present disclosure relates to use of the compounds as described herein as an intermediate in a method for isolating an ultra-high purity cannabinoid from a cannabis plant material or an extract thereof.

In another aspect, the present disclosure relates to a method for making cannabinoid salt compounds from a cannabis plant material or an extract thereof. In an embodiment, the method comprises: mixing a cannabis plant material or an extract thereof with a suitable solvent to provide a first mixture that comprises a cannabinoid; and mixing the first mixture with a base to provide a second mixture, the second mixture comprising a precipitate that comprises a cannabinoid salt compound.

In an embodiment, the methods disclosed herein may further comprise a step of separating the precipitate from at least a portion of the second composition to provide a separated precipitate.

In an embodiment, the methods disclosed herein may further comprise a step of heating the separated precipitate under reduced pressure to expel the base from the cannabinoid salt compound thereby isolating a desired cannabinoid. Therefore, in an embodiment, the compounds of the present disclosure may be used as an intermediate in a method for isolating an ultra-high purity cannabinoid from a cannabis plant material or an extract thereof

The present disclosure is based on extensive research and development directed at overcoming at least some of the current impediments to advancing the state of the art in isolating cannabinoids in high-purity forms. The present disclosure advances this field with the provision of cannabinoid salt compounds that are isolated from a suitable extraction solvent by precipitation with a suitable base. The cannabinoid salt compounds may then be utilized in a tandem decarboxylation/base-expulsion process that yields the cannabinoid in neutral form.

More generally, the methods of the present disclosure provide cannabinoid salt compounds that may: (i) reduce the need for downstream purification of the associated cannabinoid; (ii) minimize solvent incorporation into consumable cannabinoid products; and/or (iii) provide cannabinoid materials in solid forms that may be inaccessible using conventional methods. Accordingly, the methods of the present disclosure may be better suited to large-scale processes for preparing recreational and/or pharmaceutical preparations that contain cannabinoids.

Without being bound by any particular theory, the cannabinoid salt compounds of the present disclosure are more stable and more easily isolated from the cannabis plant material or extract thereof than a neutral cannabinoid compound alone. For example, the cannabinoid salt compounds may generally precipitate out of the second mixture faster than a neutral cannabinoid compound. The cannabinoid salt compounds may have an increased stability (shelf life) as compared to their neutral counterparts. The cannabinoid salt compounds of the present disclosure may have improved solubility as compared to their neutral counterparts and/or other cannabinoid salts. Furthermore, the base used to form the cannabinoid salt compounds of the present disclosure is selected from compounds that are known to have low toxicity when consumed and they may provide further desirable benefits.

Other aspects and features of the methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a schematic for a method for making cannabinoid salt compounds, according to embodiments of the present disclosure.

FIG. 2 shows a high performance liquid chromatography (HPLC) chromatogram in relation to an exemplary cannabinoid salt compound (THCA-arginine) obtained by the methods disclosed herein.

FIG. 3 shows an HPLC chromatogram from experiments into the solubility of the cannabinoid salt compounds of FIG. 2 in glycerin.

FIG. 4 shows an HPLC chromatogram in relation to a cannabinoid salt compound (THCA-benzathine) according to embodiments of the present disclosure.

FIG. 5 shows an HPLC chromatogram of a cannabinoid salt compound (CBDA-arginine) according to embodiments of the present disclosure.

FIG. 6 shows an HPLC chromatogram of a cannabinoid salt compound (THCA-lysine) according to embodiments of the present disclosure.

FIG. 7 shows a solubility graph of cannabinoid salt compounds of the present disclosure in water.

FIG. 8 shows a solubility graph of a CBDA-arginine salt compound of the present disclosure in water, MCT oil, and PEG 400.

### DETAILED DESCRIPTION

An important aspect of the cannabis industry is isolating cannabinoids from a cannabis plant material or extracts thereof in an efficient and cost-effective manner. In many instances, it is desirable to isolate ultra-high purity cannabinoids from cannabis plant material or extracts thereof. It may be particularly desirable to isolate such ultra-high purity cannabinoids in a solid powder form that is easier to handle and accurately weigh than the oil that is provided by known extraction methods.

The present disclosure relates to cannabinoid salt compounds. In an embodiment, the cannabinoid salt compounds disclosed herein may be used as an intermediate in a method for isolating ultra-high purity cannabinoids from a cannabis plant material or an extract thereof.

As used herein, the term “cannabinoid salt compound” is intended to refer to an ionic compound formed by deprotonation of the carboxylic moiety of an acidic cannabinoid.

In one aspect, the present disclosure provides compounds of the Formula 1:

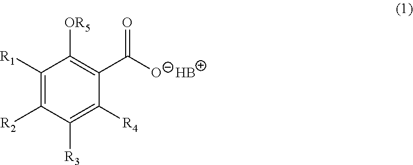

wherein R₁, R₂, R₃, R₄ and R₅ are, independently or in combination with one another, chosen so as to provide a cannabinoid, and B is a base that is of low toxicity.

As used herein, the term “base” is intended to refer to compounds comprising a lone electron pair available to bond with a proton (H⁺). The bases of the present disclosure may be capable of deprotonating an acidic hydrogen atom. By “acidic hydrogen atom” it is meant that the hydrogen atom has a tendency to be released as a proton. The tendency for a hydrogen atom to be released as a proton may be a result of being bonded to an atom or group of atoms with higher electronegativity.

As used herein, the term “low toxicity” is intended to refer to a compound suitable for consumption by a human in, for example, a recreational or pharmaceutical preparation. Toxicity is generally understood to be the degree in which a chemical substance or mixture of substances can harm humans or animals. By low toxicity, it is meant that the bases encompassed herein would require a high dosage and/or long exposure to initiate harmful effects on a human (if at all) and, therefore, are of low toxicity.

In addition to low toxicity, other desirable properties of the base may include, but are not limited to: easy expulsion from the cannabinoid salt compound to provide an ultra-high purity cannabinoid that is substantially free of the base; and potentially further pharmacologic properties, such as increasing the shelf stability of the cannabinoid salt compound, increasing the time frame under which the cannabinoid salt compound is metabolized by a subject who consumes it, or combinations thereof.

In some embodiments of the present disclosure, selecting a base having particular properties with respect to, but not limited to, pKb and boiling point, may advantageously facilitate precipitation, promote expulsion of the base, be of low toxicity and may provide further desirable pharmacologic properties. As used herein, the term “pKb” is intended to refer to negative base-10 logarithm of the base dissociation constant (Kb). The pKb value may be used to evaluate the strength of a base. In some embodiments of the present disclosure, the base has a pKb sufficiently basic to deprotonate an acidic hydrogen of an acidic cannabinoid. Those skilled in the art who have benefitted from the teachings of the present disclosure will appreciate that base strength is typically reported as the pKa of the conjugate acid in the literature and that pKb values may be calculated by EQN 1.

pKb=13.9965−pKa EQN. 1

In some embodiments, the base has a pKb of between about 0.5 and about 6.5. In some embodiments of the present disclosure, the base has a pKb of between about 3.0 and about 5.0. In some embodiments, the base has a pKb of between about 4.0 and about 5.0, such as for example certain embodiments in which the base is benzathine.

In some embodiments of the present disclosure, the base has a boiling point between about 50° C. and about 490° C. under atmospheric pressure conditions. In some embodiments of the present disclosure, the base has a boiling point between about 50° C. and about 400° C. under atmospheric pressure conditions. In some embodiments of the present disclosure, the base has a boiling point of between about 200° C. and about 375° C. under atmospheric pressure conditions. At least one advantage of the boiling points disclosed herein is that the base can later be removed by, for example, distillation. In some embodiments of the present disclosure, the base has a molecular weight for promoting precipitation. In some embodiments of the present disclosure, the base has steric properties for promoting precipitation.

In some embodiments of the present disclosure, the base comprises an amine. As used herein, the term “amine” is intended to refer to a compound containing a basic nitrogen atom with a loan pair. Amines are derivatives of ammonia, wherein one or more hydrogen atoms have been replaced by a substituent. In some embodiments of the present disclosure, the amine is a primary amine, a secondary amine, a tertiary amine, or a combination thereof. In some embodiments of the present disclosure, the amine is a basic amino acid, N′N′-dibenzylethylenediamine (benzathine), or a combination thereof.

In an embodiment, the base is an amino acid. As used herein, the term “amino acid” is intended to refer to organic compounds that combine to form proteins. Generally, an amino acid is a compound containing amino (—NH₂) and carboxyl (—COOH) functional groups, along with a side chain (R group) specific to each amino aicd, such as below:

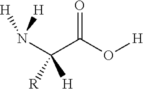

The amino acid may be obtained from natural sources or may be produced synthetically. In an embodiment, the amino acid is any one of the amino acids: arginine, histidine, lysine, aspartic acid, glutamic acid, serine, threonine, asparagine, glutamine, cysteine, selenocysteine, glycine, proline, alanine, valine, leucine, isoleucine, methionine, phenylalanine, tyrosine, tryptophan, or any combination thereof. In an embodiment, the amino acid is a modified amino acid. By “modified amino acid”, it is meant to refer to an amino acid that has undergone a chemical transformation either naturally or via synthetic means, For example and without limitation, the amino acid may be modified by hydroxylation, phosphorylation, glycosylation (e.g. O-linked glycosylation), acetylation, or oxidation.

In an embodiment, the amino acid may be either an essential amino acid (i.e. cannot be made by the human body and, therefore, must be consumed through food or otherwise provided) or a non-essential amino acid (i.e. the human body can produce the amino acid in the absence of consuming it in food). The essential amino acids are histidine, isoleucine, leucine, lysine, methionine, phenylalanine, threonine, tryptophan, and valine. Non-essential amino acids include alanine, arginine, asparagine, aspartic acid, cysteine, glutamic acid, glutamine, glycine, proline, serine, and tyrosine.

In an embodiment, the amino acid comprises an aliphatic side chain. In an embodiment, the amino acid is alanine, isoleucine, leucine, valine, or any combination thereof. In a particular embodiment, the amino acid is alanine. In a particular embodiment, the amino acid is isoleucine. In a particular embodiment, the amino acid is leucine. In a particular embodiment, the amino acid is valine.

In an embodiment, the amino acid comprises a branched side chain. In an embodiment, the amino acid is isoleucine, leucine, valine, or any combination thereof. In a particular embodiment, the amino acid is isoleucine. In a particular embodiment, the amino acid is leucine. In a particular embodiment, the amino acid is valine.

In an embodiment, the amino acid comprises an aromatic side chain. In an embodiment, the amino acid is phenylalanine, tryptophan, tyrosine, or any combination thereof. In a particular embodiment, the amino acid is phenylalanine. In a particular embodiment, the amino acid is tryptophan. In a particular embodiment, the amino acid is tyrosine.

In an embodiment, the amino acid comprises a polar neutral side chain. In an embodiment, the amino acid is asparagine, cysteine, glutamine, serine, threonine, or any combination thereof. In a particular embodiment, the amino acid is asparagine. In a particular embodiment, the amino acid is cysteine. In a particular embodiment, the amino acid is glutamine. In a particular embodiment, the amino acid is serine. In a particular embodiment, the amino acid is threonine.

In an embodiment, the amino acid comprises an electrically charged acidic side chain. In an embodiment, the amino acid is aspartic acid, glutamic acid, or a combination thereof. In a particular embodiment, the amino acid is aspartic acid. In a particular embodiment, the amino acid is glutamic acid.

In an embodiment, the amino acid comprises an electrically charged basic side chain. In an embodiment, the amino acid is arginine, histidine, lysine, or any combination thereof. In a particular embodiment, the amino acid is arginine. In a particular embodiment, the amino acid is histidine. In a particular embodiment, the amino acid is lysine.

In an embodiment, the amino acid comprises a sulfur-containing side chain. In an embodiment, the amino acid is cysteine, methionine, or a combination thereof. In a particular embodiment, the amino acid is cysteine. In a particular embodiment, the amino acid is methionine.

In an embodiment, the amino acid comprises an amidic side chain. In an embodiment, the amino acid is asparagine, glutamine, or any combination thereof. In a particular embodiment, the amino acid is asparagine. In a particular embodiment, the amino acid is glutamine.

In an embodiment, the amino acid comprises an essential dietary micronutrient. In an embodiment, the essential dietary micronutrient is selenium. In an embodiment, the amino acid is selenocysteine.

An advantage of using amino acids for the compounds and methods disclosed herein is that trace amounts of residual base will not be harmful for human consumption.

In some embodiments, the amine is a cyclic amine. Cyclic amines are a sub-category of secondary or tertiary amines, wherein the basic nitrogen atom is contained within a heterocycle that may be further substituted, or not.

In some embodiments, the amine comprises more than one basic nitrogen atom, for example benzathine. In some embodiments of the present disclosure, the base is an active pharmaceutical ingredient with basic properties such as, but not limited to, lidocaine, adrenaline, meglumine and benzathine.

In an embodiment, the base is benzathine. Benzathine is used as an ingredient in pharmaceutical preparations of penicillin, such as phenoxymethylpenicillin and benzathine benzylpenicillin. Benzathine has low toxicity to mammals and it is used to stabilize the penicillin and to modulate its release into a subject's body when injected.

The compound of Formula 1 disclosed herein comprises R₁, R₂, R₃, R₄ and R₅ that are independently or in combination with one another, chosen so as to provide a cannabinoid. As used herein, the term “cannabinoid” refers to: (i) a chemical compound belonging to a class of secondary compounds commonly found in plants of genus cannabis; (ii) synthetic cannabinoids and any enantiomers thereof; and/or (iii) one of a class of diverse chemical compounds that may act on cannabinoid receptors such as CB1 and CB2.

The most notable cannabinoid of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In some embodiments of the present disclosure, the cannabinoid is a compound found in a plant, e.g., a plant of genus cannabis, and is sometimes referred to as a phytocannabinoid. In some embodiments of the present disclosure, the cannabinoid is a compound found in a mammal, sometimes called an endocannabinoid. In some embodiments of the present disclosure, the cannabinoid is made in a laboratory setting, sometimes called a synthetic cannabinoid. In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid incudes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Within the context of this disclosure, the term “acidic cannabinoid”, is intended to refer to the carboxylic acid derivative of a particular cannabinoid.

Examples of acidic cannabinoids include, but are not limited to: cannabigerolic acid (CBGA), cannabigerolic acid monomethylether (CBGAM), cannabigerovarinic acid (CBGVA), cannabichromenic acid (CBCA), cannabichromevarinic acid (CBCVA), cannabidiolic acid (CBDA), cannabidivarinic acid (CBDVA), tetrahydrocannabinolic acid A (THCA-A), tetrahydrocannabinolic acid B (THCA-B), tetrahydrocannabinolic acid-C4 (THCA-C4), tetrahydrocannabivarinic acid (THCVA), tetrahydrocannabiorcolic acid (THCA-C1), Δ8-tetrahydrocannabinolic acid (Δ8-THCA), Δ9-tetrahydrocannabinolic acid (Δ9-THCA), cannabicyclolic acid (CBLA), cannabielsoic acid-A (CBEA-A), cannabielsoic acid-B (CBEA-B), cannabinolic acid (CBNA), cannabivarin acid (CBNVA), cannabinodiol acid (CBNDA), cannabivarinodiol acid (CBNDVA), cannabicyclol acid (CBLA), cannabicyclovarin acid (CBLVA), cannabielsoin acid (CBEA), cannabivarinselsoin acid (CBEVA), cannabitriol acid (CBTA), or cannabicitran acid.

Elimination of CO₂ from acidic cannabinoids provides what are known in the art as decarboxylated or “neutral” cannabinoids. Neutral cannabinoids can also be provided by synthetic methods. In some embodiments of the present disclosure, the ultra-high purity cannabinoid is a neutral cannabinoid.

Examples of neutral cannabinoids include, but are not limited to: cannabigerol (CBG), cannabigerol monomethylether (CBGM), cannabigerovarin (CBGV), cannabichromene (CBC), cannabichromevarin (CBCV), cannabidiol (CBD), Δ6-cannabidiol (Δ6-CBD), cannabidiol monomethylether (CBDM), cannabidiol-C4 (CBD-C4), cannabidivarin (CBDV), cannabidiorcol (CBD-C1), tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), tetrahydrocannbinol-C4 (THC-C4), tetrahydrocannabivarin (THCV), Δ8-tetrahydrocannabivarin (Δ8-THCV), Δ9-tetrahydrocannabivarin (Δ9-THCV), tetrahydrocannabiorcol (THC-C1), Δ7-cis-iso-tetrahydrocannabivarin, cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabino-C2 (CBN-C2), cannabiorcol (CBN-C1), cannabinodiol (CBND), cannabinodivarin (CBDV), cannabitriol (CBT), 11-hydroxy-Δ9-tetrahydrocannabinol (11-OH-THC), 11-nor-9-carboxy-Δ9-tetrahydrocannabinol, ethoxy-cannabitriolvarin (CBTVE), 10-ethoxy-9-hydroxy-Δ6a-tetrahydrocannabinol, cannabitriolvarin (CBTV), 8,9 dihydroxy-Δ6a(10a)-tetrahydrocannabinol (8,9-Di-OH-CBT-C5), dehydrocannabifuran (DCBF), cannabifuran (CBF), cannabichromanon (CBCN), cannabicitran, 10-oxo-Δ6a(10a)-tetrahydrocannabinol (OTHC), Δ9-cis-tetrahydrocannabinol (cis-THC), cannabiripsol (cbr), 3,4,5,6-tetrahydro-7-hydroxy-alpha-alpha-2-trimethyl-9-n-propyl-2,6-methano-2h-1-benzoxocin-5-methanol (OH-iso-HHCV), trihydroxy-delta-9-tetrahydrocannabinol (triOH-THC), yangonin, epigallocatechin gallate, dodeca-2e, 4e, 8z, 10z-tetraenoic acid isobutylamide, hexahydrocannibinol, and dodeca-2e,4e-dienoic acid isobutylamide.

Within the context of this disclosure, where reference is made to a particular cannabinoid without specifying if it is acidic or neutral, each of the acid and/or decarboxylated forms are contemplated as both single molecules and mixtures.

As used herein, the term “THC” refers to tetrahydrocannabinol. “THC” is used interchangeably herein with “Δ9-THC”. As used herein, unless explicitly stated, the term “THCA” includes THCA-A, THCA-B, THCA-C4, or any combination thereof.

In some embodiments of the present disclosure, the cannabinoid is THC (Δ9-THC), Δ8-THC, trans-Δ10-THC, cis-Δ10-THC, THCA, THCV, Δ8-THCV, Δ9-THCV, CBD, CBDA, CBDV, CBDVA, CBC, CBCA, CBCV, CBG, CBGV, CBN, CBNV, CBND, CBNDV, CBE, CBEV, CBL, CBLV, CBT, or cannabicitran.

Structural formulae of cannabinoids of the present disclosure include the following:

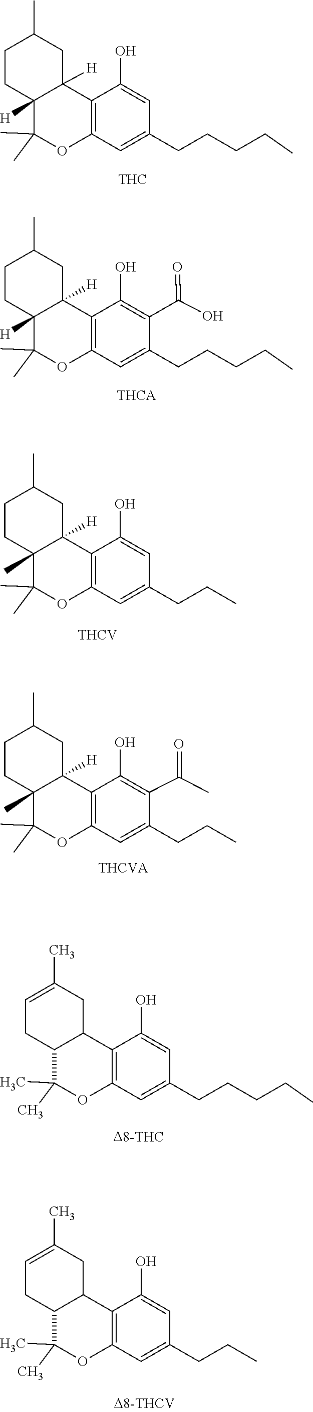

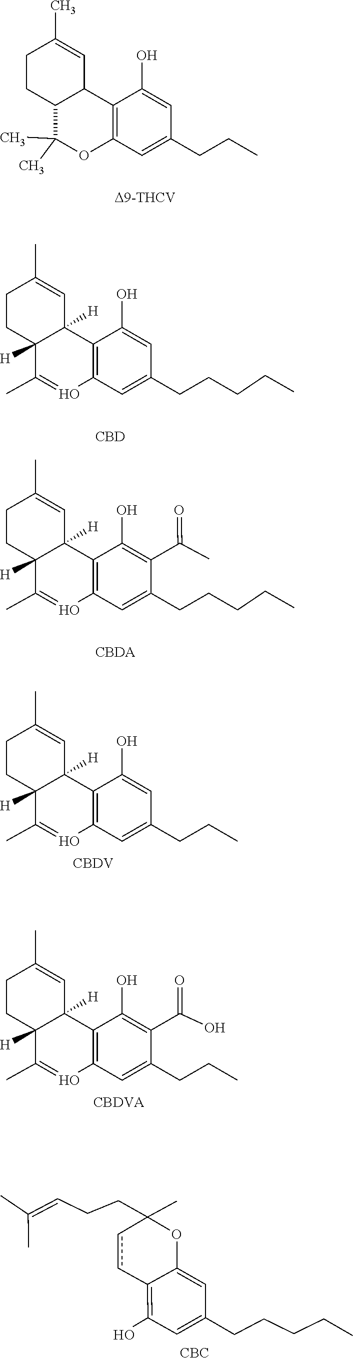

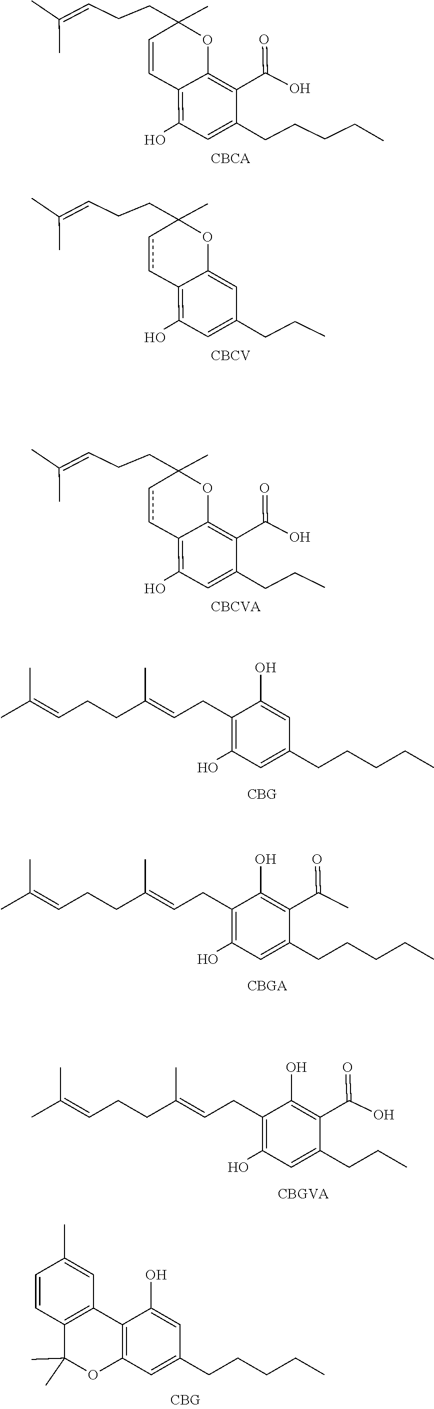

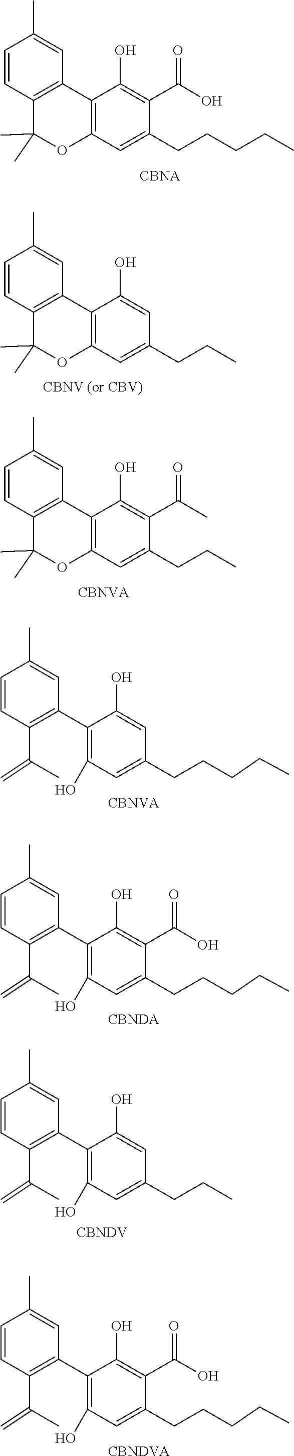

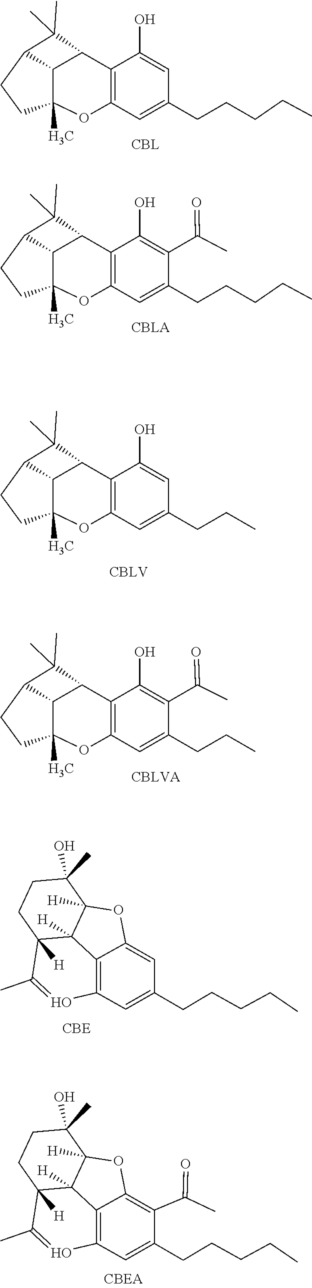

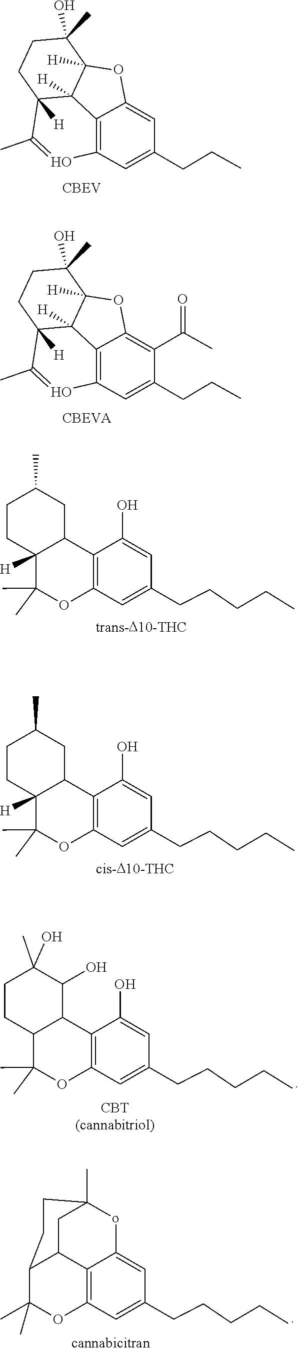

In a particular embodiment, the cannabinoid of the compound of Formula I is derived from any acidic cannabinoid including without limitation those described herein. As used herein, “derived from” means that prior to the mixing with a base the cannabinoid was in its acidic form.

In an embodiment, the cannabinoid of the compound of Formula I is derived from cannabigerolic acid (CBGA), cannabigerolic acid monomethylether (CBGAM), cannabigerovarinic acid (CBGVA), cannabichromenic acid (CBCA), cannabichromevarinic acid (CBCVA), cannabidiolic acid (CBDA), cannabidivarinic acid (CBDVA), tetrahydrocannabinolic acid (THCA), tetrahydrocannabivarinic acid (THCVA), tetrahydrocannabiorcolic acid (THCA-C1), Δ8-tetrahydrocannabinolic acid (Δ8-THCA), Δ9-tetrahydrocannabinolic acid (Δ9-THCA), cannabicyclolic acid (CBLA), cannabielsoic acid-A (CBEA-A), cannabielsoic acid-B (CBEA-B), cannabinolic acid (CBNA), cannabivarin acid (CBNVA), cannabinodiol acid (CBNDA), cannabivarinodiol acid (CBNDVA), cannabicyclol acid (CBLA), cannabicyclovarin acid (CBLVA), cannabielsoin acid (CBEA), cannabivarinselsoin acid (CBEVA), cannabitriol acid (CBTA), or cannabicitran acid.

In some embodiments of the present disclosure, the cannabinoid of the compound of Formula I is derived from CBGA.

In some embodiments of the present disclosure, the cannabinoid of the compound of Formula I is derived from CBDA.

In some embodiments of the present disclosure, the cannabinoid of the compound of Formula I is derived from THCA.

In some embodiments of the present disclosure, the ultra-high purity cannabinoid is THC (Δ9-THC), Δ8-THC, trans-Δ10-THC, cis-Δ10-THC, CBD, CBC, CBG, CBL, CBN, CBT, or cannabicitran.

In some embodiments of the present disclosure, the ultra-high purity cannabinoid is THC, CBD or CBG.

In some embodiments of the present disclosure, the ultra-high purity cannabinoid is THC.

In some embodiments of the present disclosure, the ultra-high purity cannabinoid is CBD.

In some embodiments of the present disclosure, the ultra-high purity cannabinoid is CBG.

The cannabinoid salt compounds disclosed herein can be prepared from a cannabis plant material or extract thereof, or a cannabinoid-containing composition. As will be appreciated by those skilled in the art, the cannabis plant material or extract thereof and the cannabinoid-containing composition can be selected to produce mixtures that comprise desired cannabinoid salt compounds. For example, selecting a cannabis plant material or extract that is rich in THCA will produce at least THCA-based salt compounds. Selecting a cannabis plant material or extract thereof that is rich in CBDA will produce at least CBDA-based salt compounds.

As used herein, the term “cannabis plant material” is intended to refer to any part of a cannabis plant, including without limitation: flower, stem, leaves, trichome, seed, or a combination thereof. Cannabis is a genus of flowering plants in the family Cannabaceae. The cannabis plant may be of any species, including without limitation cannabis sativa, cannabis indica, or cannabis ruderalis. In some embodiments of the present disclosure, the cannabis plant is of the species cannabis sativa. In some embodiments of the present disclosure, the cannabis plant is of the species cannabis indica. In some embodiments of the present disclosure, the cannabis plant is hemp. Hemp is a strain of the cannabis sativa species. Typically, hemp is characterised as having a low THC content. In an embodiment herein, hemp contains less than 0.3% w/w THC. In an embodiment herein, hemp contains less than 0.2% w/w THC.

As used herein, the term “or extract thereof” is intended to refer to a cannabis extract product derived from a cannabis plant material that contains cannabinoids in concentrated and/or purified form. In select embodiments of the present disclosure, the cannabis extract is obtained from a method known in the art. In select embodiments of the present disclosure, the cannabis extract is a cannabis oil.

As used herein, the term “cannabinoid-containing composition” is intended to refer to a composition that comprises at least one acidic cannabinoid and is not a cannabis plant material or extract thereof as described herein. In an embodiment, the cannabinoid-containing composition is a partially or a fully synthetic composition. By “a partially or a fully synthetic composition”, it is meant that some or all of the components in the composition are provided by chemical synthesis.

In an embodiment, the cannabis plant material or extract thereof is rich in THCA, the base is benzathine, and the cannabinoid salt compound is a monocationic salt as in Formula 2 below:

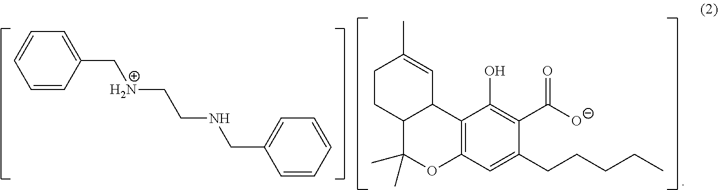

As used herein, the term “monocationic salt” is intended to refer to a salt comprising a cation bearing a single positive charge that is balanced by a cannabinoid carboxylate bearing a single negative charge. As used herein, the term “cannabinoid carboxylate” is intended to refer to an acidic cannabinoid having the carboxylic acid moiety deprotonated.

In an embodiment, the cannabis plant material or extract thereof is rich in THCA, the base is benzathine, and the cannabinoid salt compound is a dicationic salt, as in Formula 3 below:

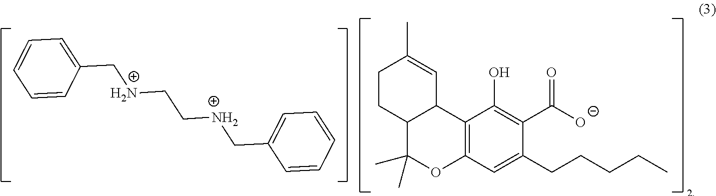

As used herein, the term “dicationic salt” is intended to refer to a salt comprising a cation bearing two positive charges that are balanced by two cannabinoid carboxylates, each bearing a single negative charge.

In an embodiment, the cannabis plant material or extract thereof is rich in THCA, the base is benzathine, and precipitate includes a mixture comprising the compound of Formula 2 and the compound of Formula 3.

In an embodiment, the cannabis plant material or extract thereof is rich in CBDA, the base is benzathine, and the cannabinoid salt compound is a monocationic salt as in, as in Formula 4 below:

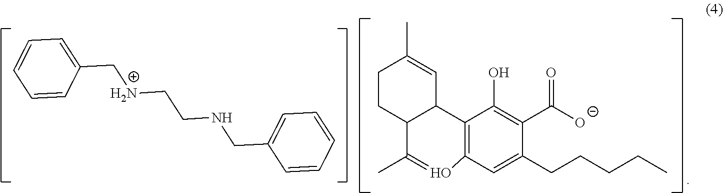

In an embodiment, the cannabis plant material or extract thereof is rich in CBDA, the base is benzathine, and the cannabinoid salt compound is a dicationic salt, as in Formula 5 below:

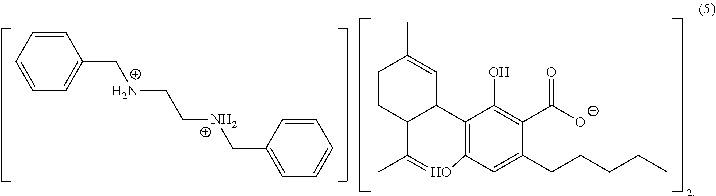

In an embodiment, the cannabis plant material or extract thereof is rich in CBDA, the base is benzathine, and the cannabinoid salt compound includes a mixture of both of the compounds of Formula 4 and Formula 5.

In an embodiment, the present disclosure provides a mixture comprising a combination of any of the compound of Formula 2, Formula 3, Formula 4, and Formula 5. In an embodiment, the cannabis plant material or extract thereof is rich in both THCA and CBD and the precipitate includes a mixture comprising a combination of any of the compound of Formula 2, the compound of Formula 3, the compound of Formula 4 and the compound of Formula 5.

In an embodiment, the mixture comprises the compound of Formula 2 and the compound of Formula 4.

In an embodiment, the mixture comprises the compound of Formula 2, the compounds of Formula 3 and further comprises one or both of the compound of Formula 4 and the compound of Formula 5.

In an embodiment, the mixture comprises the compound of Formula 4, the compound of Formula 5, and one or both of the compound of Formula 2 and the compound of Formula 3.

In an embodiment, the present disclosure provides a composition comprising the compounds disclosed herein.

The cannabinoid salt compounds disclosed herein may advantageously be in a solid form and may be stable for ease of storage, transport and dispensing. Subsequently, either in the short term or the longer term, the cannabinoid salt compounds can be subjected to a decarboxylation and base-expulsion step. For example, heating the cannabinoid salt compounds under reduced pressure may expel the base and CO₂ and provide a decarboxylated or “neutral” cannabinoid in an ultra-high purity form.

In another aspect, the present disclosure provides a method for making a cannabinoid salt compound. The method comprises mixing a cannabis plant material or extract thereof with a solvent to provide a first mixture that comprises a cannabinoid, and mixing the first mixture with a base to provide a second mixture that includes a precipitate comprising a cannabinoid salt compound (e.g. Scheme 1, step (i)). The method further comprises separating the precipitate from at least a portion of the second mixture to provide a separated precipitate comprising the cannabinoid salt compound.

Some embodiments of the present disclosure relate to making a cannabinoid salt compound from a cannabinoid-containing composition. The method comprises mixing a cannabinoid-containing composition with a solvent to provide a first mixture that comprises a cannabinoid, and mixing the first mixture with a base to provide a second mixture that includes a precipitate comprising a cannabinoid salt compound (e.g. Scheme 1, Step (i)). The methods further comprise separating the precipitate from at least a portion of the second mixture to provide a separated precipitate.

As used herein, the terms “cannabis composition” or “first composition” or “first mixture” are intended to refer to a composition provided by the mixing step, the composition comprising the solvent and one or more cannabinoids from the cannabis plant material dissolved therein. In some embodiments of the present disclosure, the cannabis composition comprises an acidic cannabinoid. In some embodiments, the cannabis composition may further comprise other components of the cannabis plant material that are soluble in the solvent. Non-limiting examples of other components of the cannabis plant material that may be soluble in the solvent include terpenes or flavonoids.

The methods of the present disclosure may further comprise heating the separated precipitate under reduced pressure to expel the base from the cannabinoid salt compound thereby isolating a final product that comprises an ultra-high purity cannabinoid (e.g. Scheme 1, Step (ii)). Thus, select embodiments of the present disclosure relate to a method for isolating an ultra-high purity cannabinoid from a cannabis plant material or extract thereof, or from a cannabinoid-containing composition.

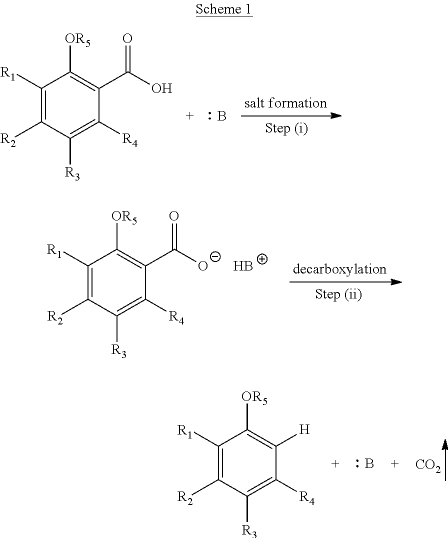

As used herein, the term “ultra-high purity cannabinoid” is meant to refer to a composition comprising a single cannabinoid in percentages close to 100%. In the context of the present disclosure, when it is said that that an ultra-high purity cannabinoid has a purity of a certain percent, it is meant that the cannabinoid is present in the composition in quantities of that percent. In some embodiments of the present disclosure, the ultra-high purity cannabinoid has a purity of at least about 98%.

In Scheme 1 above, each of R₁, R₂, R₃, R₄ and R₅ may, independently or in combination with one another, be chosen so as to provide a cannabinoid selected from any of the cannabinoids as defined herein. B represents a base that is selected to provide desirable properties to the cannabinoid salt compound produced in Step (i) above. Examples of desirable properties and non-limiting exemplary bases are discussed elsewhere herein.

The step of mixing may be by any suitable means to combine the cannabis plant material with the solvent such that at least a portion of the cannabinoids in the cannabis plant material is extracted into the solvent. In some embodiments of the present disclosure, the cannabis plant material is contacted with a solvent in a solvent-based extraction process known in the art. In an embodiment, different extraction temperatures may be used and the temperature may be dependant upon the plant material, desired cannabinoid, solvent, or any combination thereof.

As used herein, the term “solvent” is meant to refer to a substance that dissolves a solute (e.g. one or more cannabinoids). In some embodiments of the present disclosure, the solvent is a liquid. In some embodiments of the present disclosure, the solvent is a supercritical fluid. In some embodiments of the present disclosure, the solvent is a gas. In some embodiments of the present disclosure, the solvent comprises a non-polar liquid. In some embodiments of the present disclosure, the solvent is a solvent with a C6-C8 alkyl chain that may be substituted, or not. In some embodiments of the present disclosure, the solvent comprises hexane, cyclohexane, heptane, octane, isooctane, isopropyl alcohol, or a combination thereof. In some embodiments of the present disclosure, the solvent is hexane, cyclohexane, heptane, octane, isooctane, isopropyl alcohol or a combination thereof. In some embodiments of the present disclosure, the solvent is heptane. In some embodiments, the solvent is an alcohol. In some embodiments, the alcohol is ethanol, isopropyl alcohol, or a combination thereof. In some embodiments of the present disclosure, the solvent is isopropyl alcohol. In some embodiments of the present disclosure, the solvent is heptane.

In some embodiments of the present disclosure, mixing the cannabis plant material with the solvent comprises a solvent:cannabis plant material ratio of between about 30:1 and about 2:1 on a weight basis. In some embodiments of the present disclosure, the solvent:cannabis plant material ratio is about: 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 and about 1:1 on a weight basis. The term “about” is used herein to include at least a 10% deviation in either direction so that, for example, about 2:1 includes between 1.8:1 and 2.2:1). In some embodiments where the solvent:cannabis plant material ratio is about 2:1 on a weight basis, the ratio may be provided by first combining the solvent and cannabis plant material in a 1:1 ratio to provide a pre-wet material followed by combining the pre-wet material with sufficient solvent to provide the 2:1 solvent:cannabis plant material ratio.

In some embodiments, the mixing of the cannabis plant material with the solvent can occur at a temperature of between about −50° C. and about 23° C. or between about −30° C. and about 23° C. or between about −20° C. and about 23° C. or between about −10° C. and about 23° C.

In some embodiments, the method may further comprise separating the cannabis composition from the cannabis plant material after the mixing of the cannabis plant material with the solvent. The cannabis plant material may be separated from the cannabis composition by any suitable means. In some embodiments of the present disclosure, the separating comprises one or both of filtration and centrifugation. In some embodiments of the present disclosure, the separation comprises one or both of gravity filtration and vacuum filtration.

In an embodiment, the method comprises mixing the cannabis composition with a base to provide a precipitate and a solution. The base may be any of the bases disclosed elsewhere herein. The cannabis composition and base may be mixed by any suitable means so that the cannabis composition and base can react to form the precipitate, for example in a reaction vessel. In some embodiments of the present disclosure, the mixing of the cannabis composition with a base comprises stirring, agitation, or a combination thereof. In some embodiments of the present disclosure, the precipitate comprises a cannabinoid salt compound, which may also be referred to as a cannabinoid-base adduct or a cannabinoid salt.

In some embodiments of the present disclosure, the base may be mixed in an amount of about five molar equivalents with respect to the acidic hydrogen atoms in the cannabis composition. As used herein, the term “equivalent” is intended to refer to the amount of a substance required to fully react with an amount of another substance. In the context of the present disclosure, an equivalent of base is an amount of base required to react with all of the acidic hydrogens in the cannabis composition. In some embodiments of the present disclosure, the base is combined in between about 5 equivalents and about 1.5 equivalents with respect to the acidic hydrogens. In some embodiments of the present disclosure, the cannabis composition and base are mixed for a time between about 36 hours and less than an hour. In some embodiments of the present disclosure, the mixing time is between 24 hours and about half an hour. In some embodiments of the present disclosure, the time is about 24 hours.

In some embodiments, the method comprises separating the precipitate from the solution. As used here, the term “solution” is intended to refer to a solvent comprising at least one solute. In some embodiments of the present disclosure, the solute may be compounds from the cannabis plant material. In some embodiments of the present disclosure, the solution is substantially free of acidic cannabinoids. In some embodiments of the present disclosure, the separating comprises filtering. Without limitation, the filtering may for example be gravity filtration, suction or vacuum filtration, or centrifugal filtration. In some embodiments of the present disclosure, the filtering is by using a filtering funnel under vacuum. In some embodiments of the present disclosure, the filtration is performed through a semi-permeable filter paper or a finely porous glass.

In some embodiments of the present disclosure, the method further comprises heating the separated precipitate under reduced pressure to expel the base from the cannabinoid salt compound and provide an ultra-high purity cannabinoid. In these embodiments, the cannabinoid salt compound is an intermediate compound. As used herein, the term “intermediate compound” is intended to refer to the fact that the cannabinoid salt was prepared in the course of the chemical synthesis but it is not itself the final product.

In an embodiment, the heating is at a temperature between about 100° C. and about 180° C. In some embodiments of the present disclosure, the heating is at a temperature between about 120° C. and about 170° C., and more particularly between about 140° C. and about 160° C. In some embodiments of the present disclosure, the heating is at a temperature of 140° C., 141° C., 142° C., 143° C., 144° C., 145° C., 146° C., 147° C., 148° C., 149° C., 150° C., 151° C. 152° C., 153° C., 154° C., 155° C., 156° C., 157° C., 158° C., 159° C., or 160° C.

In some embodiments of the present disclosure, the heating is for a period of time between about 2 hours and about 6 hours. In some embodiments of the present disclosure, heating is for about 4 hours. In some embodiments of the present disclosure, heating is for a period of time of about 3.5 hours, about 3.75 hours, about 4 hours, about 4.25 hours, or about 4.5 hours.

In some embodiments of the present disclosure, the heating is at a temperature between about 150° C. and about 170° C. for between about 2 hours and about 6 hours. In some embodiments of the present disclosure, the heating is at a temperature of about 160° C. for about 4 hours. In some embodiments of the present disclosure, the reduced pressure can be between about 0.05 mbar and about 10 mbar.

In some embodiments of the present disclosure, the heating comprises removing the expelled base to provide an ultra-high purity cannabinoid. In some embodiments of the present disclosure, the expelled base is removed by distillation. In some embodiments of the present disclosure, the distillation is by vacuum distillation, fractional distillation, or short path distillation. In some embodiments of the present disclosure, the distillation is by short path distillation at between about 150° C. and about 160° C. at >0.05 mbar for about three hours. In some embodiments of the present disclosure, the ultra-high purity cannabinoid is isolated after the heating. In some embodiments of the present disclosure, the ultra-high purity cannabinoid isolated after heating is THC that is a solid at temperatures below about 0° C.

In some embodiments of the present disclosure, the ultra-high purity cannabinoid has a purity of at least 99% of the ultra-high purity cannabinoid. In some embodiments of the present disclosure, the ultra-high purity cannabinoid has a purity of at least 99.5%. In some embodiments of the present disclosure, the ultra-high purity cannabinoid has a purity of at least about 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, or 99.9%. In some embodiments of the present disclosure, the other component of the final product may be a different cannabinoid, the base, or a combination thereof.

In another aspect of the present disclosure, there is provided a method for isolating an ultra-high purity cannabinoid from a cannabinoid-containing composition, the method comprising: mixing the cannabinoid-containing composition with a base and a solvent to provide a precipitate and a solution; separating the precipitate from at least a portion of the solution; and heating the precipitate under reduced pressure to expel the base and provide the ultra-high purity cannabinoid, wherein the heating comprises removing the base from the ultra-high purity cannabinoid. In these embodiments, the cannabinoid-containing composition is provided by a method other than presently disclosed and the base is a base selected from those disclosed elsewhere herein.

In some embodiments of the present disclosure, the cannabinoid-containing composition comprises tetrahydrocannabinol acid (THCA), tetrahydrocannabivarin acid (THCVA), cannabidiol acid (CBDA), cannabidivarin acid (CBDVA), cannabichromene acid (CBCA), cannabichromevarin acid (CBCVA), cannabigerol acid (CBGA), cannabigerovarin acid (CBGVA), cannabinol acid (CBNA), cannabivarin acid (CBNVA), cannabinodiol acid (CBNDA), cannabivarinodiol acid (CBNDVA), cannabicyclol acid (CBLA), cannabicyclovarin acid (CBLVA), cannabielsoin acid (CBEA), cannabivarinselsoin acid (CBEVA), cannabitriol acid (CBTA), cannabicitran acid, or any combination thereof.

In some embodiments of the present disclosure, mixing the cannabinoid-containing composition with the solvent comprises a solvent:cannabinoid-containing composition ratio of ratio of between about 30:1 and about 2:1 on a weight basis. In some embodiments of the present disclosure, the solvent:cannabinoid-containing composition ratio is about: 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 and about 1:1 on a weight basis. In some embodiments of the present disclosure, the solvent:cannabinoid-containing composition ratio is between about 25:1 and about 15:1 on a weight basis. In some embodiments of the present disclosure, the solvent:cannabinoid-containing ratio is about 20:1 on a weight basis. The mixing may be by any suitable means so that the cannabinoid-containing composition and the base can react to form the precipitate. In some embodiments of the present disclosure, the mixing comprises stirring.

The solvent may be any solvent as described elsewhere herein. In some embodiments, the solvent comprises hexane, cyclohexane, heptane, octane, isooctane, isopropyl alcohol, or a combination thereof. In some embodiments of the present disclosure, the solvent is heptane. In some embodiments of the present disclosure, the solvent is isopropyl alcohol. In some embodiments of the present disclosure, mixing the cannabinoid-containing composition with the solvent comprises a solvent:cannabinoid-containing composition ratio of between about 30:1 and about 2:1 on a weight basis.

In some embodiments, the base comprises an amine such as those described elsewhere herein. In some embodiments of the present disclosure, the amine is N, N′-dibenzylethylenediamine. In some embodiments of the present disclosure, the amine is an amino acid as described elsewhere herein. In some embodiments of the present disclosure, the amino acid is arginine.

In some embodiments of the present disclosure, the base may be contacted in an amount of about 2 equivalents with respect to the acidic hydrogen atoms in the cannabinoid-containing composition. In some embodiments of the present disclosure, the base is combined in about 1.5 equivalents with respect to the acidic hydrogen atoms. In some embodiments of the present disclosure, the cannabinoid-containing composition and base are contacted for a time between about 10 minutes and about 2 hours. In some embodiments of the present disclosure, the time is between 30 minutes and 1.5 hours. In some embodiments of the present disclosure, the time is about 1 hour.

In some embodiments, the method for isolating an ultra-high purity cannabinoid from a cannabinoid-containing composition comprises separating the precipitate from at least a portion of the solution. In some embodiments of the present disclosure, the precipitate comprises a cannabinoid salt. In some embodiments of the present disclosure, the separating comprises filtration or centrifugation. Without limitation, the filtration may for example be gravity filtration, suction or vacuum filtration, or centrifugal filtration. In some embodiments of the present disclosure, the filtration comprises gravity filtration or vacuum filtration.

In some embodiments of the present disclosure, the method for isolating an ultra-high purity cannabinoid from a cannabinoid-containing composition comprises heating under reduced pressure. The heating under reduced pressure may be at the temperatures, times, and pressures disclosed elsewhere herein. In some embodiments of the present disclosure, the heating is at a temperature between about 150° C. and about 170° C. for between about 2 hours and about 6 hours. In some embodiments of the present disclosure, the heating is at a temperature of about 160° C. for about 4 hours.

In some embodiments of the present disclosure, the heating comprises removing the expelled base to provide the ultra-high purity cannabinoid. In some embodiments of the present disclosure, the expelled base is removed by distillation. In some embodiments of the present disclosure, the distillation is by vacuum distillation, fractional distillation, or short path distillation. In some embodiments of the present disclosure, the distillation is by short path distillation. In some embodiments of the present disclosure, the short path distillation is between about 150° C. and about 160° C. at >0.05 mbar for about three hours. In some embodiments of the present disclosure, the ultra-high purity cannabinoid is isolated after the heating.

In some embodiments of the present disclosure, the ultra-high purity cannabinoid has a purity of at least about 98%. In some embodiments of the present disclosure, the ultra-high purity cannabinoid is THC (Δ9-THC), Δ8-THC, trans-Δ10-THC, cis-Δ10-THC, THCV, Δ8-THCV, Δ9-THCV, CBD, CBDA, CBDV, CBDVA, CBC, CBCA, CBCV, CBG, CBGV, CBN, CBNV, CBND, CBNDV, CBE, CBEV, CBL, CBLV, CBT, or cannabicitran. In some embodiments of the present disclosure, the ultra-high purity cannabinoid is THC. In some embodiments of the present disclosure, the ultra-high purity cannabinoid is CBD.

In some embodiments, the methods for isolating ultra-high purity cannabinoids disclosed herein further comprise reusing at least a portion of the expelled base in the step of mixing the composition with the base. In some embodiments of the present disclosure, the methods disclosed herein further comprise recovering at least a portion of the base following the separating of the precipitate. As used herein, the term “recovering” is intended to refer to obtaining a material in its original form after it has been through at least a portion of the method. A non-limiting example of recovering a portion of the base is distillation of the solution after the separating of the precipitate to provide the base.

In some embodiments, the methods disclosed herein further comprise recovering at least a portion of the solvent following the separating of the precipitate. In some embodiments of the present disclosure, recovering at least a portion of the base, at least a portion of the solvent or both, is following the separating of the precipitate. In some embodiments of the present disclosure, the recovering is by distillation. In some embodiments of the present disclosure, at least a portion of the base is recovered from the heating step. In some embodiments, a portion of the recovered base, solvent or both may be reused in the steps of the methods disclosed herein. In some embodiments of the present disclosure, at least a portion of the base from the heating step is reused in the step of mixing the composition with the base. In a non-limiting example, the recovered base may be used as the base in the mixing step in the method for isolating a high purity cannabinoid from a cannabis plant material or cannabinoid-containing composition.

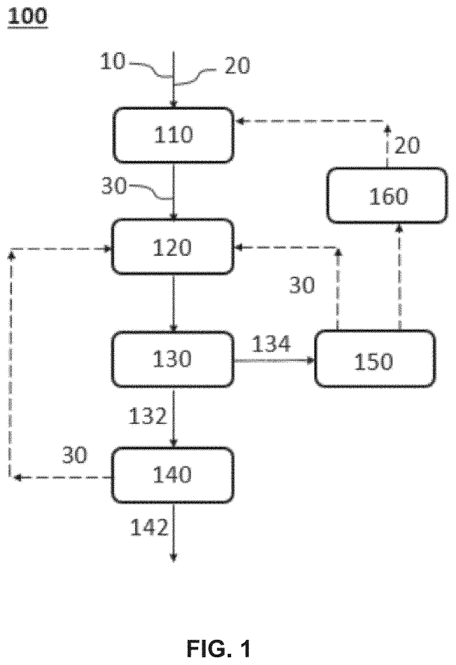

Embodiments of the present disclosure will now be detailed by reference to FIG. 1, which shows a schematic of a flow diagram for a method 100 for isolating an ultra-high purity cannabinoid from a cannabis plant material in accordance with the methods disclosed herein.

The method 100 comprises the following steps: mixing (110) the cannabis plant material 10 with a solvent 20 to form a first mixture; mixing (120) the first mixture with a base 30 to provide a precipitate 132 that comprises a cannabinoid salt compound, and a second composition 134; separating (130) the precipitate 132 from at least a portion of the second composition 134; and heating (140) the precipitate 132 under reduced pressure to expel the base 30 from the cannabinoid salt compound thereby providing the ultra-high purity cannabinoid 142. The base 30 expelled in the heating (140) may be used in the mixing step (120), or not. The method 100 may further comprise a step of recovering (150) the base 30 from the second composition 134. The recovered base 30 may be used in the mixing step 120, or not. The method 100 may further comprise a step (160) of recovering the solvent 20. The recovered solvent 20 may be used in the mixing step 110, or not.

### EXAMPLES

### Example 1

About 40 grams of THCA-rich fresh marijuana flower was mixed with isopropyl alcohol in a volume-to-weight ratio of 20:1 (solvent:flower) to provide a first mixture. The mixing proceeded at −20° C. To the first mixture was added one equivalent of arginine to provide a second mixture. The second mixture was stirred overnight at about room temperature, which resulted in a precipitate that comprised a cannabinoid salt compound of Formula 6:

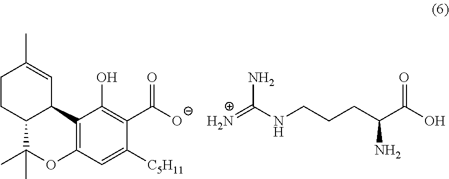

The solvent was evaporated and the precipitate comprising the cannabinoid salt compound of Formula 6 was washed with tert-butyl-methyl ether (TBME) to provide beige coloured crystals of a THCA-arginine salt.

Analysis of the cannabinoid salt compounds exemplified herein was performed using high performance liquid chromatography (HPLC). Formic acid was used in sample preparation and, therefore, the purity of the prepared cannabinoid salt compound was based on the acidic cannabinoid content (e.g. CBDA, THCA) in the resulting HPLC chromatogram.

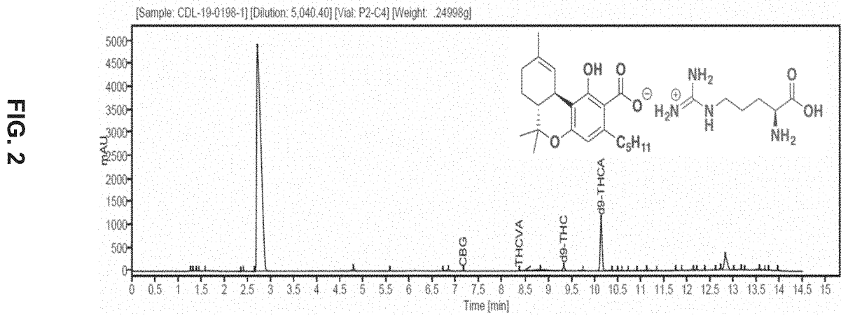

FIG. 2 shows an HPLC chromatogram for the THCA-arginine salt having about 90% w/w of THCA content. There were other acidic cannabinoids present, including CBDA (about 1% w/w) and CBGA (about 1% w/w).

### Example 2

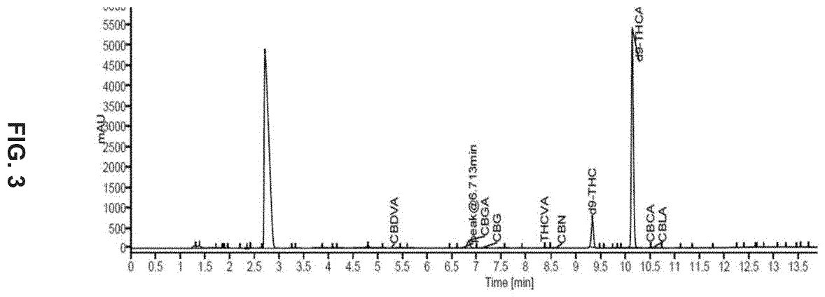

To assess the suitability of using THCA-arginine salts in recreational and pharmaceutical preparations, the solubility of THCA-arginine salts in glycerin was examined. Briefly, about 0.1 g of the THCA-arginine salt prepared by Example 1 was added to about 2.555 g of glycerin. Initially, this mixture was viscous but then the salt dissolved in the glycerin. FIG. 3 shows an HPLC chromatograph of the salt-glycerin mixture. About 3% w/w of the salt was equal to about 2% w/w of THCA in the glycerin by HPLC. At the time of the compositional analysis, about 3% of the THCA was observed to have decarboxylated to THC.

### Example 3

About 40 grams of THCA-rich fresh marijuana flower was mixed with isopropyl alcohol in a volume-to-weight ratio of 20:1 (solvent:flower) to provide a first mixture. The extraction step proceeded at −20° C. and the volume of the first mixture was reduced by about 50-100mL by evaporation.

To the first mixture was added benzathine at about a 1.5 molar equivalent to provide a second mixture. The second mixture was stirred overnight at about room temperature, which resulted in a precipitate that included cannabinoid salt compounds that had formed from the acidic cannabinoid and the benzathine base such as, for example, the compound of Formula 2 and the compound of Formula 3:

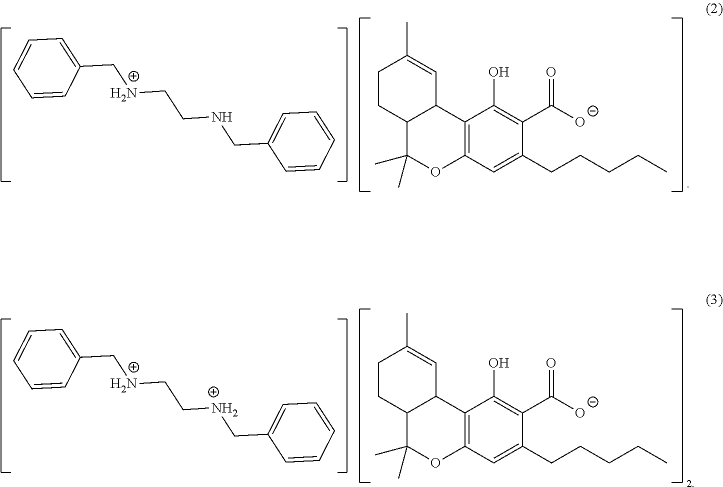

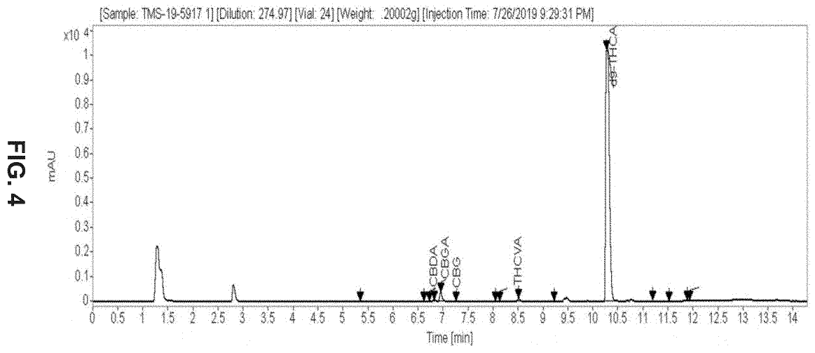

The solvent was evaporated and the cannabinoid salt compounds crystallized. The crystals were washed with tert-butyl-methyl ether (TBME) to provide beige coloured crystals of THCA-benzathine salt. FIG. 4 shows an HPLC chromatogram for the THCA-benzathine salt that was found to have about 74.45% THCA content, which corresponds to greater than 95% THCA-benzathine salt purity and a 2:1 THCA:benzathine ratio in the salt (compound 3).

### Example 4

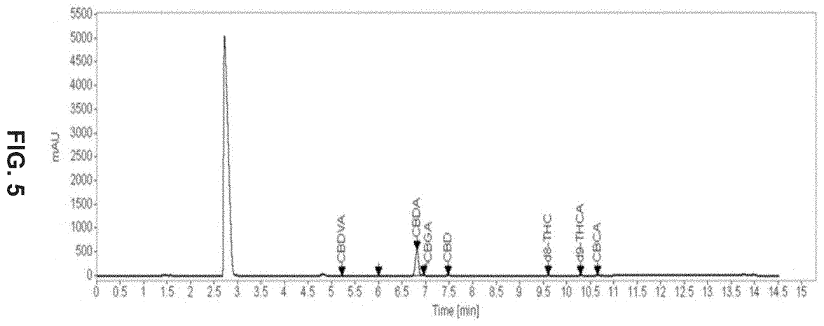

In an ethanol solution, a CBDA extract was mixed with arginine (1 molar equivalent to CBDA) at room temperature. The resulting solution was evaporated to dryness using a rotary evaporator. Heptane was added and the reaction mixture was stirred. The mixture was filtered under low vacuum. The filter cake was washed two times with heptane and left to air dry to provide a cream-coloured product. FIG. 5 shows an HPLC chromatogram of the product. Based on CBDA content, the CBDA-arginine salt was determined to be obtained in greater than 95 w/w % purity.

### Example 5

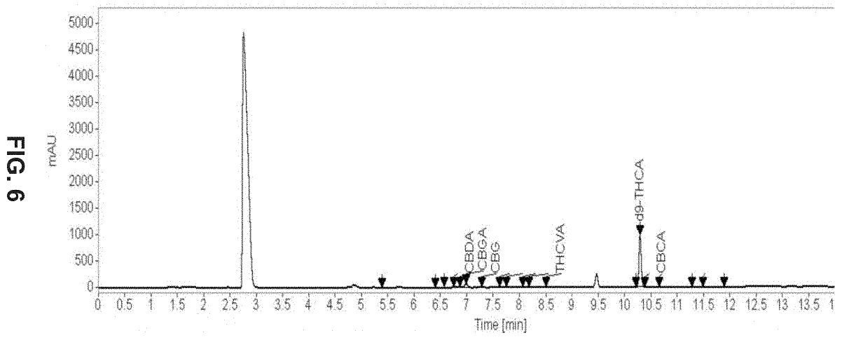

A THCA resin (about 55% THCA) was dissolved in ethanol under heating, and L-lysine was added and mixed. The resulting solution was evaporated to dryness using a rotary evaporator. Heptane was added and the reaction mixture was stirred. The solution was filtered and washed with heptane. The obtained filter cake was dried in air to provide a dried product. FIG. 6 shows an HPLC chromatogram of the product. Based on THCA content, the THCA-lysine salt was determined to be obtained in greater than 73 w/w % purity.

### Example 6

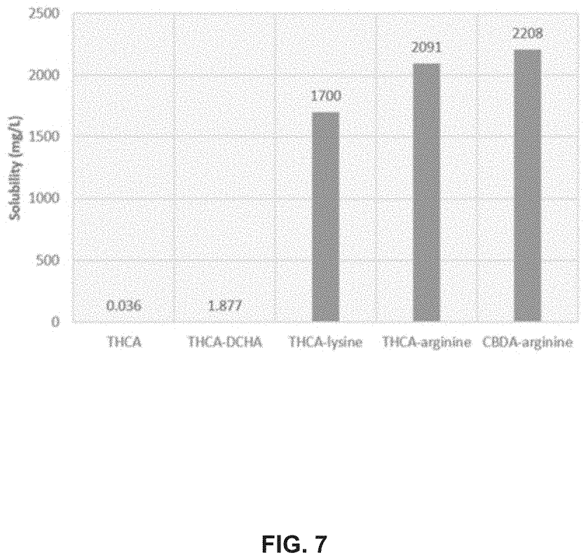

Solubility in water of THCA, a THCA-dicyclohexyl amine (DCHA) salt, and the prepared salts of the present disclosure: THCA-arginine salt (Example 1), CBDA-arginine salt (Example 4) and THCA-lysine salt (Example 5), was investigated. In a test tube, 50 mg of the respective cannabinoid salt compound was suspended in 8 mL of deionized water and vortexed. The solution was filtered with a syringe equipped with 0.45 micron filter. HPLC was used to determine the quantity of dissolved cannabinoids. As shown in FIG. 7, THCA and the THCA-DCHA salt were virtually insoluble in water, whereas the amino acid salts of the present disclosure advantageously showed significant improvement in water solubility.

### Example 7

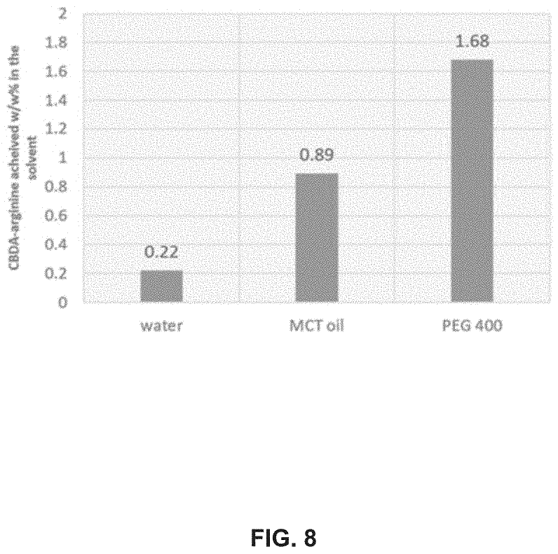

Solubility of the CBDA-arginine salt of the present disclosure (Example 4) was evaluated in MCT oil and PEG 400. 50 mg of the CBD-arginine salt was suspended in 8 mL of either the MCT oil or PEG 400 and stirred for 12 h at room temperature. The solution was centrifuged for 5 min at 2800 rpm and filtered. The filtrate was analyzed by HPLC to determine the quantity of dissolved cannabinoids. As shown in FIG. 8, the CBDA-arginine salt showed increased solubility in MCT oil and PEG 400 as compared to water. This may be particularly advantageous in preparing products employing these types of carrier oils.

### Example 8

CBDA and the CBDA-arginine salt of the present disclosure (Example 4) were evaluated in a simulated gastric fluid (SGF) assay. In a microcentrifuge tube, 626.5 μL of a solution containing 1.1× assay buffer (37.6 mM NaCI, pH 1.2-1.5) was diluted with 70 μL of a 10× pepsin solution (80,000 U/mL in milliQ water). The resulting solution was incubated at 37° C. and 1,200 rpm in an orbital mixer for 5 min, prior to the addition of 3.5 μL of CBDA or CBDA-arginine salt, respectively, in 2 mM, DMSO. The sample was incubated under the same conditions. At the specified time points, 150 μL aliquots of the sample were transferred to microcentrifuge tubes containing 24 μL of quenching solution (0.5 M NaHCO₃). After vortexing for 5 seconds, 348 μL of protein precipitation solution containing an internal standard (25 μM Glyburide, ACN) was added. The tube was vortexed again for 20 seconds and stored in ice. Finally, the tubes were centrifuged at 5,000×g and 4° C. for 15 min. The percentage remaining CBDA or CBDA-arginine salt, respectively, compared to time zero, was quantified in the supernatant by HPLC/LC-MS or LC-MS/MS. It was found that under these experimental conditions both the CBDA and the CBDA-arginine salt of the present disclosure had a half-life of about 19.7 min in a gastric environment.

### Example 9

THCA and the THCA-arginine salt of the present disclosure (Example 1) were evaluated in a SGF assay in accordance with the procedure described in Example 8. It was found that under these experimental conditions the THCA had a half-life of about 12.2 min and the THCA-arginine salt had a half-life of about 14.4 min.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

1. A compound of Formula 1: 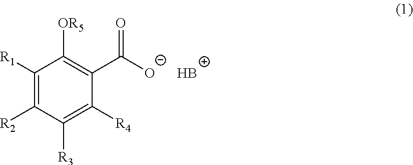
wherein R₁, R₂, R₃, R₄ and R₅ are, independently or in combination with one another, chosen so as to provide a cannabinoid, and
B is a base that is of low toxicity.

2. The compound of claim 1, wherein the cannabinoid is cannabigerolic acid (CBGA), cannabigerolic acid monomethylether (CBGAM), cannabigerovarinic acid (CBGVA), cannabichromenic acid (CBCA), cannabichromevarinic acid (CBCVA), cannabidiolic acid (CBDA), cannabidivarinic acid (CBDVA), tetrahydrocannabinolic acid (THCA), tetrahydrocannabivarinic acid (THCVA), tetrahydrocannabiorcolic acid (THCA-C1), Δ8-tetrahydrocannabinolic acid (Δ8-THCA), Δ9-tetrahydrocannabinolic acid (Δ9-THCA), cannabicyclolic acid (CBLA), cannabielsoic acid-A (CBEA-A), cannabielsoic acid-B (CBEA-B), cannabinolic acid (CBNA), cannabivarin acid (CBNVA), cannabinodiol acid (CBNDA), cannabivarinodiol acid (CBNDVA), cannabicyclol acid (CBLA), cannabicyclovarin acid (CBLVA), cannabielsoin acid (CBEA), cannabivarinselsoin acid (CBEVA), cannabitriol acid (CBTA), or cannabicitran acid.

3. The compound of claim 1, wherein the cannabinoid is cannabigerolic acid (CBGA), cannabidiolic acid (CBDA), or tetrahydrocannabinolic acid (THCA).

4. The compound of claim 1, wherein the base is benzathine.

5. The compound of claim 1, which is of Formula 2:

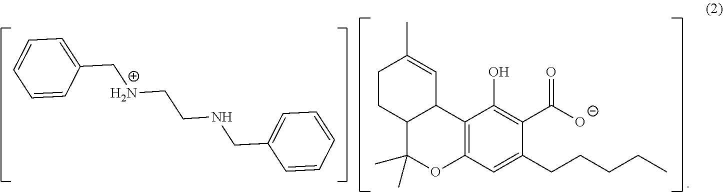

6. The compound of claim 1, which is of Formula 3:

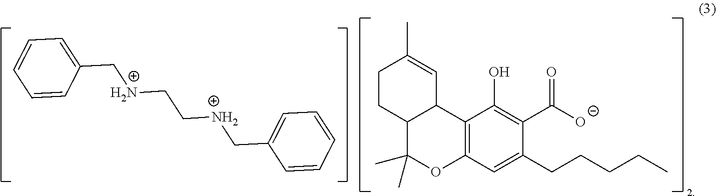

7. The compound of claim 1, which is of Formula 4:

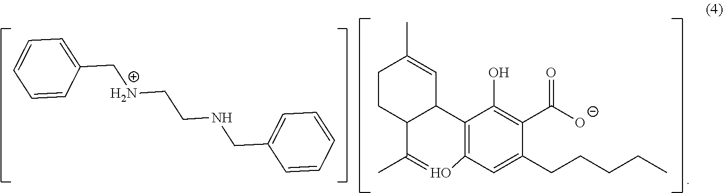

8. The compound of claim 1, which is of Formula 5:

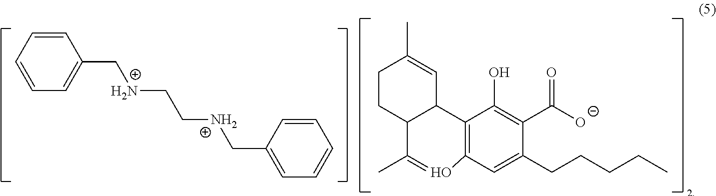

9. The compound of claim 1, wherein the base is an amino acid.

10.-12. (canceled)

13. The compound of claim 9, wherein the amino acid is arginine, histidine, lysine, aspartic acid, glutamic acid, serine, threonine, asparagine, glutamine, cysteine, selenocysteine, glycine, proline, alanine, valine, leucine, isoleucine, methionine, phenylalanine, tyrosine, tryptophan, or any combination thereof.

14. The compound of claim 9, wherein the amino acid is arginine, histidine, or lysine.

15.-24. (canceled)

25. A method for preparing an ultra-high purity cannabinoid, the method comprising:
mixing a cannabis plant material or an extract thereof with a solvent and a base to provide a mixture comprising a compound of Formula 1: 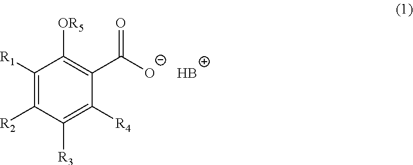
wherein R₁, R₂, R₃, R₄ and R₅ are, independently or in combination with one another, chosen so as to provide a cannabinoid,
separating the compound of Formula 1 from at least a portion of the mixture, and
obtaining an ultra-high purity cannabinoid from the compound of Formula 1.

26. The method of claim 25, wherein the step of separating the compound of Formula 1 comprises isolating a precipitate that comprises the compound of Formula 1.

27. The method of claim 25, wherein the step of obtaining the ultra-high purity cannabinoid comprises heating the compound of Formula 1.

28. The method of claim 25, wherein the cannabinoid is cannabigerolic acid (CBGA), cannabigerolic acid monomethylether (CBGAM), cannabigerovarinic acid (CBGVA), cannabichromenic acid (CBCA), cannabichromevarinic acid (CBCVA), cannabidiolic acid (CBDA), cannabidivarinic acid (CBDVA), tetrahydrocannabinolic acid (THCA), tetrahydrocannabivarinic acid (THCVA), tetrahydrocannabiorcolic acid (THCA-C1), Δ8-tetrahydrocannabinolic acid (Δ8-THCA), Δ9-tetrahydrocannabinolic acid (Δ9-THCA), cannabicyclolic acid (CBLA), cannabielsoic acid-A (CBEA-A), cannabielsoic acid-B (CBEA-B), cannabinolic acid (CBNA), cannabivarin acid (CBNVA), cannabinodiol acid (CBNDA), cannabivarinodiol acid (CBNDVA), cannabicyclol acid (CBLA), cannabicyclovarin acid (CBLVA), cannabielsoin acid (CBEA), cannabivarinselsoin acid (CBEVA), cannabitriol acid (CBTA), or cannabicitran acid.

29. The method of claim 25, wherein the cannabinoid is cannabigerolic acid (CBGA), cannabidiolic acid (CBDA), or tetrahydrocannabinolic acid (THCA).

30. The method of claim 25, wherein the base is benzathine.

31. The method of claim 25, wherein the compound of Formula 1 is:
a compound of Formula 2: 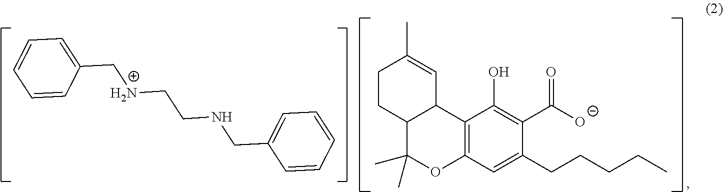
a compound of Formula 3: 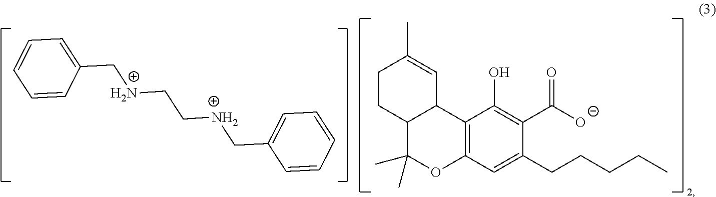
a compound of Formula 4: 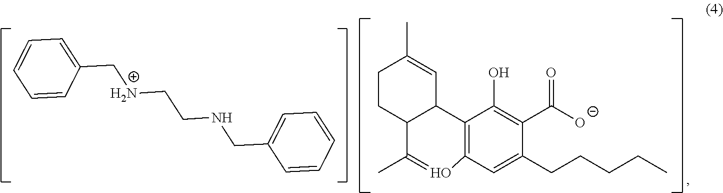
a compound of Formula 5: 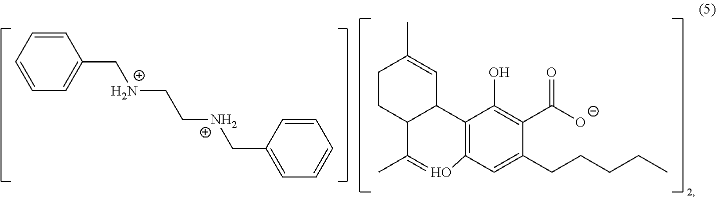
or any combination thereof.

32. The method of claim 25, wherein the base is an amino acid.

33. The method of claim 32, wherein the amino acid is arginine, histidine, or lysine.
